(19) United States
(12) Patent Application Publication
(10) Pub. No.: US 2009/0191292 A1
(43) Pub. Date: Jul. 30, 2009

(54) High essential oil and eugenol yielding cultivar of ocimum sanctum 'CIM-AYU'

(75) Inventors: Raj Kishori Lal, Lucknow (IN); Suman Preet Singh Khanuja, Lucknow (IN); Arun Kumar Agnihotri, Lucknow (IN); Hari Om Misra, Lucknow (IN); Ajit Kumar Shasany, Lucknow (IN); Ali Arif Naqvi, Lucknow (IN); Om Parkash Dhawan, Lucknow (IN); Alok Kalra, Lucknow (IN); Janak Raj Bahl, Lucknow (IN); Mahendra Pandurang Darokar, Lucknow (IN)

Correspondence Address: FOLEY AND LARDNER LLP;SUITE 500, 3000 K STREET NW, WASHINGTON, DC, 20007, US

(73) Assignee: COUNCIL OF SCIENTIFIC AND INDUSTRIAL RESEARCH

(21) Appl. No.: 11/825,452

(22) Filed: Jul. 6, 2007

Related U.S. Application Data
(63) Continuation of application No. 10/639,682, filed on Aug. 13, 2003, now abandoned.

(51) Int. Cl. A61K 36/53

(52) U.S. Cl. 424/745

(57) ABSTRACT

The present invention relates to the development of a novel high herb yielding essential oil herb variety of Krishna Tulsi (botanically known as Ocimum sanctum, from the Family—Lamiaceae, 2n=32), and hereinafter named as ‘CIM-AYU’. In particular, the invention is related to the development of a high eugenol yielding variety of Krishna Tulsi named ‘CIM-AYU’ through open pollination in the germplasm followed by recurrent progeny selection and evaluation for the yield characters of selected population for 3 years in field conditions. The selected variety is high yielding and stable in subsequent generations. This invention thus relates to the high yielding seeds, plants and plant parts of plant named ‘CIM-AYU’ and its components, to a method of producing named ‘CIM-AYU’, and to a method for producing high eugenol using ‘CIM-AYU’ as a pollinator or parent.

### CROSS-REFERENCE TO RELATED APPLICATION(S)

This application is a Continuation of U.S. application Ser. No. 10/639,682, filed Aug. 13, 2003, incorporated herein by reference in its entirety.

### LATIN NAME OF THE GENUS AND SPECIES

Ocimum sanctum

### VARIETY DENOMINATION

‘CIM-AYU’

### FIELD OF THE INVENTION

The present invention relates to the development of a novel high herb yielding essential oil herb variety of Krishna Tulsi (botanically known as Ocimum sanctum, from the Family—Lamiaceae, 2n=32), and hereinafter named as ‘CIM-AYU’. In particular, the invention is related to the development of a high eugenol yielding variety of Krishna Tulsi named ‘CIM-AYU’ through open pollination in the germplasm followed by recurrent progeny selection and evaluation for the yield characters of selected population for 3 years in field conditions. The selected variety is high yielding and stable in subsequent generations. This invention thus relates to the high yielding seeds, plants and plant parts of plant named ‘CIM-AYU’ and its components, to a method of producing named ‘CIM-AYU’, and to a method for producing high eugenol using ‘CIM-AYU’ as a pollinator or parent.

### BACKGROUND OF INVENTION

Tulsi, Ocimum sanctum, belongs to the family of Lamiaceae. Hindus know the plant as Tulasi and Surasah in Sanskrit, and Tulsi in Hindi. Due to the anti oxidant and anti ageing effects of Tulsi, Hindus use fresh leaves of this plant daily in the Panchamrut/Charanamrut drink after puja. Other commonly used names are Haripriya, dear to Vishnu, and Bhutagni, destroyer of demons. Tulsi is Divinity. It is regarded not merely as a utilitarian Godsend, as most sacred plants are viewed to be, but as an incarnation of the Goddess Herself. A plethora of Puranic legends and village stories relate how Tulsi came to grow and be worshiped on Earth. The classic Hindu myth, Samudramathana, the “Churning of the Cosmic Ocean,” explains that Vishnu spawned Tulsi from the turbulent seas as a vital aid for all mankind. More common are legends that describe how the Goddess Herself came to reside on Earth as Tulsi.

The Tulsi leaf, when eaten, can control thirst, and so was invaluable to weary travellers. Soon, the plant acquired a religious significance, and became essential in worship. Apart from religious importance, the Tulsi has several medicinal properties. It is rich in carbohydrates, fiber, phosphorous, calcium, protein, iron, beta-carotene, vitamins B1 and B2 and in aromatic oils. It is good for colds and coughs, indigestion, stomach pain and diarrhea. Nausea, ulcers, ringworm and asthma can also be effectively treated with Tulsi. It is said to lower blood sugar and increase lactation. The oil is used in antiperspirants and in fly and mosquito repellents.

Keeping in mind the traditional importance of Tulsi, together with the need for developing a better plant type having high herb, essential yield characters combined with a consistent high yield of phenyl propanoid “Eugenol” in the essential oil throughout all seasonal yields was considered and evaluated in the planned breeding and selection process which was undertaken by the inventors at the farm of CIMAP, Lucknow, India, to develop the new variety ‘CIM-AYU’. Generally, in growing conditions in India, the high eugenol yielding genotypes do not produce eugenol during the rainy season. Therefore, the primary objective of the present invention was to develop a new Ocimum sanctum cultivar possessing a high yield of essential oils especially eugenol, during the rainy season. To develop such a high yielding cultivar, recurrent selection was undertaken for the genotypes producing higher eugenol during the rainy season. Further to establish high yielding essential oil characteristics recurrent backcrossing and selection were undertaken which finally resulted in a plant cultivar named ‘CIM-AYU’ of O. sanctum. The new plant ‘CIM-AYU’ produced a high amount of essential oils, especially eugenol during the rainy season. Thus, during the process of selection, emphasis was placed on selecting a genotype producing high eugenol during the rainy season, which resulted in the new plant cultivar named ‘CIM-AYU’.

### DETAILED DESCRIPTION

1. Breeding History:

Mass and recurrent selections can be used to improve populations of either self- or cross-pollinating crops. A genetically variable population of heterozygous individuals is either identified or created by inter-crossing several different parents. The best plants are selected based on individual superiority, outstanding progeny, or excellent combining ability. The selected plants are inter-crossed to produce a new population in which further cycles of selection are continued.

2. Selection for Three Years:

In the present invention which was carried out under the genetic improvement programme of Tulsi (Ocimum sanctum), the diverse germplasm/genetic stocks of Krishna Tulsi were collected from Utter Pradesh, Bihar, Madhya Pradesh, Orissa, Uttranchal, Gujarat, Maharashtra, Jammu and Kashmir, the West Bangal states of India. The germplasm were grown in plots of 1.0 m×10.0 m size plots with 50 cm×50 cm spacing randomly. The seeds collected from individual plants were bulked and germinated in the next season (May) and transferred to the main field for evaluation (in June). Randomly 2500 plants were evaluated for better plant types with high herb and oil yield, out of which 10 plants were selected. The seeds from these plants were again bulked and planted in the next season out of which 10 plants were selected again. The same process was repeated in the next year and ultimately 10 plant types were taken for preliminary evaluation.

3. Evaluation Trials:

Ten promising genetic stocks along with a check (control) named STA-1 were evaluated in an Initial Evaluation Trial (IET). The evaluations were carried out in randomised block design (RBD) with two replications in 0.50 m² plots for each treatment during 2000-2001. Four best performing selections, KRT-2, SHTS-3, OTF-4, and STN-5 from IET and one control check (check) STA-1 were evaluated in a Bench Scale Trial (BST). Table 1 provides the mean performance data of promising strains in BST yield trials for herb and oil yield in Ocimum sanctum.

TABLE 1

| | | BST (RBD, reps 3, plot size- 12.24 sqm) | | | | |
|---|---|---|---|---|---|---|
| | | Mean herb yield | | | | |
| | | (kg/plot) | | | | Oil yield |
| | | Complete | Fresh | | Oil content (%) | (g/plot) |
| S. No | Entries | plant | leaves | Dry leaves | Dry leaves | Dry leaves |
| 1. | KRT-2 | 34.20 | 9.15 | 2.31 | 0.705 | 16.27 |
| 2. | STAS-3 | 34.31 | 8.99 | 1.99 | 0.628 | 12.45 |
| 3. | OTF-4 | 11.63 | 3.71 | 0.92 | 0.383 | 3.50 |
| 4. | STN-5 | 16.74 | 6.72 | 1.48 | 0.478 | 7.04 |
| 5. | STA-1 | 12.76 | 3.51 | 0.89 | 0.225 | 2.01 |
| | (CHECK) | | | | | |
| | CD_{(5%)} | 0.39 | 0.08 | 0.04 | 0.034 | 0.51 |
| | CD_{(1%)} | 0.55 | 0.11 | 0.05 | 0.048 | 0.71 |

Further evaluations were carried out in randomised block design (RBD) with four replications in 12.24 m² plots for each treatment during year 2001-2002. Finally, the three most promising selections KRT-2, STAS-3 and STN-5 along with check STA-1 were evaluated in a Pilot Scale Trial (PST) in 2002-2003 (84 m² for each treatment). Table 2 provides the mean performance data of promising strains in PST yield trials for herb and oil yield in Ocimum sanctum.

TABLE 2

| | | PST (plot size- 84.0 sqm) | | | | | | |
|---|---|---|---|---|---|---|---|---|
| | | Mean herb yield | | | | | | Oil yield |
| | | (kg/plot) | | | Oil content | Herb yield (ql/ha) | | (kg/ha) |
| | | Complete | Fresh | Dry | (%) | Complete | Dry | Dry |
| S. No | Entries | plant | leaves | leaves | Dry leaves | plant | leaves | leaves |
| 1. | KRT-2 | 168.00 | 53.25 | 13.31 | 0.70 | 200.00 | 15.85 | 110.95 |
| | ‘CIM-AYU’ | | | | | | | |
| 2. | STAS-3 | 152.04 | 55.87 | 12.04 | 0.64 | 181.00 | 14.33 | 91.71 |
| 3. | OTF-4 | — | — | — | — | — | — | — |
| 4. | STN-5 | 113.39 | 46.20 | 10.84 | 0.40 | 134.99 | 12.90 | 51.0 |
| 5. | STA-1 | 113.40 | 42.42 | 10.92 | 0.23 | 135.00 | 13.00 | 29.90 |
| | (CHECK) | | | | | | | |
| | CD_{(5%)} | — | — | — | — | — | — | — |
| | CD_{(1%)} | — | — | — | — | — | — | — |

On an average, the elite strain KRT-2 registered its superiority over all other selections including check STA-1 for whole plant herb, dry leaves and oil yield of better quality per unit area. The elite strain KRT-2 was named as variety ‘CIM-AYU’. Table 3 provides an comparison of the oil composition of variety ‘CIM-AYU’ (KRT-2) and check STA-1 of Ocimum sanctum.

TABLE 3

| S. No. | Constituents in oil | CIM-AYU (KRT-2) | STA- 1 (Check) |
|---|---|---|---|
| 1. | β-pinene | 0.13 | — |
| 2. | α-pinene | 0.19 | 0.05 |
| 3. | Camphene | 0.09 | 0.08 |
| 4. | Sabinene | 0.03 | 0.13 |
| 5. | Limonene | 0.05 | 0.78 |
| 6. | p-Cymene | — | 0.11 |
| 7. | Cineole | 0.21 | 2.50 |
| 8. | Linalool | 0.05 | 1.92 |
| 9. | Eugenol | 83.56 | 40.42 |
| 10. | β-elemene | 7.47 | 14.11 |
| 11. | β-caryophyllene | 6.93 | 9.07 |
| 12. | Germacrene-D | — | 16.65 |

During screening and experimentation, individual strains were maintained in seed plots with an isolation distance of 500 m² and seeds obtained from these seed plots were used in growing the plants for evaluation. During the evaluation trials 10 plants from each strain were evaluated through profiling the population DNA and comparing among each other for maintenance of purity and stability through generations. The plants when grown in isolation as mentioned are self pollinated and maintain the stability and purity as observed from the morphological, essential oil and DNA profiles.

### BRIEF DESCRIPTION OF THE ACCOMPANYING PHOTOGRAPHS

FIG. 1: shows photograph of the plant of CIM-AYU.

FIG. 2: shows Lane: 1 Hind III digest molecular weight markers: Lane 2 to 13: Profiles with AAATCGGAGC (SEQ ID NO: 1), TGCGCGATCG (SEQ ID NO: 4), AACGTACGCG (SEQ ID NO: 5), CGGGATCCGC (SEQ ID NO: 9), GCGAATTCCG (SEQ ID NO: 10), CCCTGCAGGC (SEQ ID NO: 11), CCAAGCTTGC (SEQ ID NO: 12), AAGATAGCGG (SEQ ID NO: 15), GGATCTGAAC (SEQ ID NO: 16), TTGTCTCAGG (SEQ ID NO: 17), GGACTCCACG (SEQ ID NO: 19), CACCCTGCGC (SEQ ID NO: 7) primers.

### DETAILED DESCRIPTION OF THE INVENTION

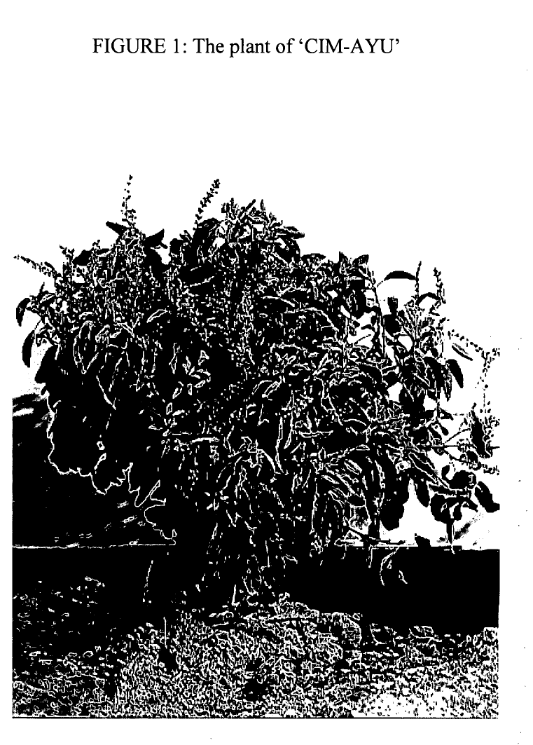

According the present invention relates to the strain KRT-2, subsequently named as ‘CIM-AYU’. The developed plant variety named as ‘CIM-AYU’, consistently showed a higher herbage and oil content in BST and PST yield trials. The yield of the complete plant was estimated to be 200 quintals per hectare with dry leaf yield of 15.85 quintals per hectare. The total oil yield was 110.95 kilogram per hectare. All the yields (total plant, dry leaf and oil) were higher than all other strains taken for comparison. Further, the developed variety ‘CIM-AYU’ produced higher eugenol in the essential oil compared to the control variety, STA-1. During the agro-climatic condition in the rainy season in India, it is observed that the eugenol content of the leaf decreases drastically in many of the Ocimum sanctum genotypes sometimes to 0% from 80%. But in this new variety ‘CIM-AYU’, during the rainy season, the eugenol content was found to be 47% and this was observed during three consecutive growing seasons, for three consecutive years (1997 through 2000). FIG. 1 shows a photograph of the plant of CIM-AYU.

The main embodiment of the present invention relates to a new and distinct high yielding essential oil plant of Ocimum sanctum of variety Tulsi Krishna named ‘CIM-AYU’ developed through cross open pollination of diversely collected germplasm followed by recurrent progeny selection and evaluation by screening for morphology, high herbal and essential oil content and further recurrent selection for uniformity and stability for a period of three years to establish selected traits such as high herbal and essential oil content, the said plant after yield evaluation in the field having following characteristics:
(a) the said plant produces a high amount of eugenol (83% of oil) (higher than STA-1 which produces a lower amount of eugenol (40.42% of oil)) with high essential oil yield (0.7 to 0.72%) as well as herbage yield (at least fresh total plant 200 and dry leaf 15.85 quintals per hectare),
(b) the said plant possesses vigorous vegetative growth with a 66 cm×78 cm canopy area and a height of at least 75.67 cm in a maximum of 100 days,
(c) the said plant has a sturdy and erect growth habit with profuse, synchronous branching (about 17 branches per plant) (more than STA-1 which produces an average 13 branches per plant),
(d) the said plant produces a stem which is quadrangular in shape at the lower part (older part of stem) and which is round in shape at the top of the plant, which is strong and hardy in supporting the plant, and which is primarily green in color (RHS 139D), with faint purplish red pigmentation at time of maturity (RHS 79D), and with a mean thickness of 0.7 cm,
(e) the said plant has distinct molecular profile by random amplified polymorphic DNA (RAPD) using 12 random primers distinguishing the plant from the other existing varieties,
(f) the said plant produces leaves having light greenish (RHS 137C), chartaceous texture, minute surface hairs on the above, hirsute hairs below with nerves, oblong to ob-lanceolate shape with serrate margins, sub-acute to acuminate tip, obtuse base and moderately broad size (length about 4.93 cm and width about 2.73 cm),
(g) said plant produces a petiole extending to size of 2.4 cm,
(h) said plant spreads in an area of 15.8 cm², at average of full branch,
(i) the said plant produces an indefinite racemose inflorescence with 10-15 florets having light green colour (RHS 142B) in early stage and pale purple when mature (RHS 79D) with flowers arranged in whorls surrounding the peduncle at the base of the lateral leaves having pinkish white flowers (RHS 56C),
(j) the said plant is able to produce higher herbage, oil and eugenol yield per unit area as compared to other control genotypes,
(k) the said plant produces full plant herb yield 200 quintal per hectare and from fresh leaves yield 63.39 quintal per hectare to 15.85 quintal per hectare dry leave yield,
(l) the said plant produces at least 10 kilogram per hectare essential oil rich in eugenol (80 to 84%) the content during the growing season, and
(m) the said plant produces eugenol in the essential oil even during rainy season (at least 47%) compared to 5.0% in check and other strains.

Table 4 provides comparison data for the new variety ‘CIM-AYU’ (KRT-2) and the check variety STA-1 of Ocimum sanctum.

TABLE 4

| | Elite Strain | Check Strain |
|---|---|---|
| Attributes | ‘CIM-AYU’ (KRT-1) | STA-1 |
| Plant height (cm) | 75.67 | 63.33 |
| Days of flowering (50%) | 76 (early) | 92 (late) |
| Growth habit | Semi closed | Open |
| No. of branches/plant | 17.33 | 12.67 |
| Length of leaf (cm) | 4.93 | 4.63 |
| Width of leaf (cm) | 2.73 | 2.83 |
| Whole plant herb yield | 200.00 | 135.00 |
| (ql/ha) | | |
| Dry leaf yield (q/ha) | 15.85 | 13.00 |
| Oil content (%) | 0.70 | 0.23 |
| Oil yield (Kg/ha) | 110.95 | 29.90 |
| Colour of stem | Light Green | Violet |
| Colour of leaves | Light Green | Light Violet |
| Eugenol (%) | 83.56 | 40.42 |
| β-Elemene (%) | 7.47 | 14.11 |
| β-Caryophyllene (%) | 6.93 | 9.07 |

Uniformity and Stability

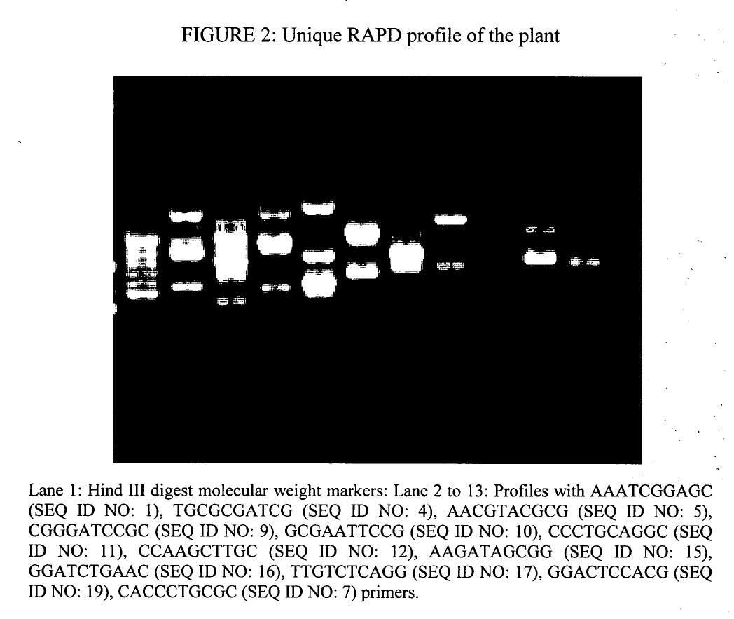

When the uniform plant population of CIM-AYU is grown in isolation with minimum isolation distance of 500 m from other genotypes of Ocimum sanctum, outcrossing from undesirable genotypes does not take place. Instead the population purity is being maintained through restricting pollination within the population components to maintain allelic balance within the population in nature of equilibrium. In the present invention the purity of the plant variety was maintained by growing the plant population with an isolation distance of 500 m from any other genotypes of Ocimum sanctum. The stability of the plant population was checked through pooled DNA profiling using 20 MAP primers in subsequent generations and found to be uniform without variation. The sequences of the primers MAP01 to MAP20 were AAATCGGAGC (SEQ ID NO: 1), GTCCTACTCG (SEQ ID NO:2), GTCCTTAGCG (SEQ ID NO: 3), TGCGCGATCG (SEQ ID NO:4), AACGTACGCG (SEQ ID NO:5), GCACGCCGGA (SEQ ID NO: 6), CACCCTGCGC (SEQ ID NO: 7), CTATCGCCGC (SEQ ID NO: 8), CGGGATCCGC (SEQ ID NO: 9), GCGAATTCCG (SEQ ID NO: 10), CCCTGCAGGC (SEQ ID NO: 11), CCAAGCTTGC (SEQ ID NO: 12), GTGCAATGAG (SEQ ID NO: 13), AGGATACGTG (SEQ ID NO: 14), AAGATAGCGG (SEQ ID NO: 15), GGATCTGAAC (SEQ ID NO: 16), TTGTCTCAGG (SEQ ID NO: 17), CATCCCGAAC (SEQ ID NO: 18), GGACTCCACG (SEQ ID NO: 19), AGCCTGACGC (SEQ ID NO: 20), respectively. The primers AAATCGGAGC (SEQ ID NO: 1), TGCGCGATCG (SEQ ID NO: 4), AACGTACGCG (SEQ ID NO: 5), CGGGATCCGC (SEQ ID NO: 9), GCGAATTCCG (SEQ ID NO: 10), CCCTGCAGGC (SEQ ID NO: 11), CCAAGCTTGC (SEQ ID NO: 12), AAGATAGCGG (SEQ ID NO: 15), GGATCTGAAC (SEQ ID NO: 16), TTGTCTCAGG (SEQ ID NO: 17), GGACTCCACG (SEQ ID NO: 19), CACCCTGCGC (SEQ ID NO: 7) were used to develop the unique fingerprint pattern of the variety and the pattern was found to be consistent for three generations. These latter primers give a specific and unique DNA fingerprinting pattern for the plant named ‘CIM-AYU’. These primers therefore can be considered specific as to the cultivar ‘CIM-AYU’ for they represent the DNA patterns found only for in this plant cultivar. FIG. 2 shows Lane: 1 Hind III digest molecular weight markers: Lane 2 to 13: Profiles with AAATCGGAGC (SEQ ID NO: 1), TGCGCGATCG (SEQ ID NO: 4), AACGTACGCG (SEQ ID NO: 5), CGGGATCCGC (SEQ ID NO: 9), GCGAATTCCG (SEQ ID NO: 10), CCCTGCAGGC (SEQ ID NO: 11), CCAAGCTTGC (SEQ ID NO: 12), AAGATAGCGG (SEQ ID NO: 15), GGATCTGAAC (SEQ ID NO: 16), TTGTCTCAGG (SEQ ID NO: 17), GGACTCCACG (SEQ ID NO: 19), CACCCTGCGC (SEQ ID NO: 7) primers.

Time and Place of Development of the Variety

The variety CIM-AYU was developed at CIMAP and all the field trials were conducted at the farm of CIMAP, Lucknow, India. The selection and screening was started in the year 1997 and from the year 2000 the yield trials were undertaken till 2002.

Color references are made to the Royal Horticultural Society Colour Chart (RHS), except where general color of ordinary significance are used.

Taxonomic Description of the Ocimum Plant Named ‘CIM-AYU’

1. Genus: Ocimum
2. Species: sanctum
3. Family: Lamiaceae
4. Common name: Krishna Tulsi
5. Plant height: 75.67 cm in 100 days
6. Plant canopy: 66 cm×78 cm
7. Growth habit: Erect and sturdy main stem, profuse synchronous branching (about 17 branches per plant).
8. Stem: Round (at top of plant) to quadrangular (at lower part of plant—older part of stem) in shape, strong and hardy in strength, green (RHS 139D), faint purplish red pigmentation at the time of maturity (RHS 79D), Mean thickness 0.7 cm.
9. Leaf:
   Colour: Light green (RHS 137C) both upper and lower surface
   Texture: Chartaceous
   Surface: Minute hairs above, hirsute below and nerves
   Shape: Oblong to ob-lanceolate
   Margin: Serrate
   Tip: Sub-acute to acuminate
   Base: Obtuse
   Size: Moderately broad
   Petiole length: 2.4 cm
   Area: 15.8 cm² (Average of full branch)
   Length: 4.93 cm
   Width: 2.73 cm
10. Leaf: stem ratio (w/w): 0.84
11. Inflorescence: Indefinite racemose
   Total number of florets: 10 to 15
   Colour early stage: Light green (RHS 142B)
   Colour maturity: Pale purple (RHS 79D)
12. Flowers:
   Arranged in whorls surrounding the peduncle at the base of lateral leaves.
13. Oil content in the dry leaf (%): 0.7 to 0.72
14. Oil quality:
   Eugenol (%): 80 to 84
15. Herbage (Quintal per hectare): Fresh total plant: 200
   Dry leaf: 15.85

What is claimed:

1. A new and distinct high yielding essential oil plant of Ocimum sanctum of variety Tulsi Krishna named ‘CIM-AYU’ developed through cross open pollination of diversely collected germplasm followed by recurrent progeny selection and evaluation by screening for morphology, high herbal and essential oil content and further recurrent selection for uniformity and stability for a period of three years to establish selected traits such as high herbal and essential oil content, the said plant after yield evaluation in the field having following characteristics:
(a) the said plant produces a high amount of eugenol (83% of oil) (higher than STA-1 which produces a lower amount of eugenol (40.42% of oil)) with high essential oil yield (0.7 to 0.72%) as well as herbage yield (at least fresh total plant 200 and dry leaf 15.85 quintals per hectare),
(b) the said plant possesses vigorous vegetative growth with a 66 cm×78 cm canopy area and a height of at least 75.67 cm in a maximum of 100 days,
(c) the said plant has a sturdy and erect growth habit with profuse, synchronous branching (about 17 branches per plant) (more than STA-1 which produces an average 13 branches per plant),
(d) the said plant produces a stem which is quadrangular in shape at the lower part (older part of stem) and which is round in shape at the top of the plant, which is strong and hardy in supporting the plant, and which is primarily green in color (RHS 139D), with faint purplish red pigmentation at time of maturity (RHS 79D), and with a mean thickness of 0.7 cm,
(e) the said plant has distinct molecular profile by random amplified polymorphic DNA (RAPD) using 12 random primers distinguishing the plant from the other existing varieties,
(f) the said plant produces leaves having light greenish (RHS 137C), chartaceous texture, minute surface hairs on the above, hirsute hairs below with nerves, oblong to ob-lanceolate shape with serrate margins, sub-acute to acuminate tip, obtuse base and moderately broad size (length about 4.93 cm and width about 2.73 cm),
(g) said plant produces a petiole extending to size of 2.4 cm,
(h) said plant spreads in an area of 15.8 cm2, at average of full branch,
(i) the said plant produces an indefinite racemose inflorescence with 10-15 florets having light green colour (RHS 142B) in early stage and pale purple when mature (RHS 79D) with flowers arranged in whorls surrounding the peduncle at the base of the lateral leaves having pinkish white flowers (RHS 56C),
(j) the said plant is able to produce higher herbage, oil and eugenol yield per unit area as compared to other control genotypes,
(k) the said plant produces full plant herb yield 200 quintal per hectare and from fresh leaves yield 63.39 quintal per hectare to 15.85 quintal per hectare dry leave yield,
(l) the said plant produces at least 110 kilogram per hectare essential oil rich in eugenol (80 to 84%) the content during the growing season, and
(m) the said plant produces eugenol in the essential oil even during rainy season (at least 47%) compared to 5.0% in check and other strains.


SEQUENCE LISTING (20 sequences)

SEQ ID NO: 1 - DNA, 10 bp, Artificial Sequence
  Description of Artificial Sequence Synthetic Primer
aaatcggagc

SEQ ID NO: 2 - DNA, 10 bp, Artificial Sequence
  Description of Artificial Sequence Synthetic Primer
gtcctactcg

SEQ ID NO: 3 - DNA, 10 bp, Artificial Sequence
  Description of Artificial Sequence Synthetic Primer
gtccttagcg

SEQ ID NO: 4 - DNA, 10 bp, Artificial Sequence
  Description of Artificial Sequence Synthetic Primer
tgcgcgatcg

SEQ ID NO: 5 - DNA, 10 bp, Artificial Sequence
  Description of Artificial Sequence Synthetic Primer
aacgtacgcg

SEQ ID NO: 6 - DNA, 10 bp, Artificial Sequence
  Description of Artificial Sequence Synthetic Primer
gcacgccgga

SEQ ID NO: 7 - DNA, 10 bp, Artificial Sequence
  Description of Artificial Sequence Synthetic Primer
caccctgcgc

SEQ ID NO: 8 - DNA, 10 bp, Artificial Sequence
  Description of Artificial Sequence Synthetic Primer
ctatcgccgc

SEQ ID NO: 9 - DNA, 10 bp, Artificial Sequence
  Description of Artificial Sequence Synthetic Primer
cgggatccgc

SEQ ID NO: 10 - DNA, 10 bp, Artificial Sequence
  Description of Artificial Sequence Synthetic Primer
gcgaattccg

SEQ ID NO: 11 - DNA, 10 bp, Artificial Sequence
  Description of Artificial Sequence Synthetic Primer
ccctgcaggc

SEQ ID NO: 12 - DNA, 10 bp, Artificial Sequence
  Description of Artificial Sequence Synthetic Primer
ccaagcttgc

SEQ ID NO: 13 - DNA, 10 bp, Artificial Sequence
  Description of Artificial Sequence Synthetic Primer
gtgcaatgag

SEQ ID NO: 14 - DNA, 10 bp, Artificial Sequence
  Description of Artificial Sequence Synthetic Primer
aggatacgtg

SEQ ID NO: 15 - DNA, 10 bp, Artificial Sequence
  Description of Artificial Sequence Synthetic Primer
aagatagcgg

SEQ ID NO: 16 - DNA, 10 bp, Artificial Sequence
  Description of Artificial Sequence Synthetic Primer
ggatctgaac

SEQ ID NO: 17 - DNA, 10 bp, Artificial Sequence
  Description of Artificial Sequence Synthetic Primer
ttgtctcagg

SEQ ID NO: 18 - DNA, 10 bp, Artificial Sequence
  Description of Artificial Sequence Synthetic Primer
catcccgaac

SEQ ID NO: 19 - DNA, 10 bp, Artificial Sequence
  Description of Artificial Sequence Synthetic Primer
ggactccacg

SEQ ID NO: 20 - DNA, 10 bp, Artificial Sequence
  Description of Artificial Sequence Synthetic Primer
agcctgacgc